(19) United States
(12) Patent Application Publication
(10) Pub. No.: US 2025/0366731 A1
(43) Pub. Date: Dec. 4, 2025

(54) DEEP LEARNING FOR GADOLINIUM CONTRAST DETECTS BLOOD-BRAIN BARRIER OPENING

(71) Applicant: THE TRUSTEES OF COLUMBIA UNIVERSITY IN THE CITY OF NEW YORK, New York, NY (US)

(72) Inventors: Jia GUO, Fort Lee, NJ (US); Cheng-Chia WU, New York, NY (US); Andrew Francis LAINE, New York, NY (US); Pin-Yu LEE, New York, NY (US)

(73) Assignee: THE TRUSTEES OF COLUMBIA UNIVERSITY IN THE CITY OF NEW YORK, New York, NY (US)

(21) Appl. No.: 19/301,196

(22) Filed: Aug. 15, 2025

Related U.S. Application Data
(63) Continuation of application No. PCT/US24/15775, filed on Feb. 14, 2024.
(60) Provisional application No. 63/446,163, filed on Feb. 16, 2023.

(51) Int. Cl. A61B 5/055; A61B 5/00; G16H 30/20

(52) U.S. Cl. CPC A61B 5/055; A61B 5/0033; G16H 30/20

(57) ABSTRACT

The subject matter includes systems and methods for a deep learning technique applied to dynamic contrast-enhanced magnetic resonance imaging (DCE-MRI) scans. The disclosed aims to reduce the dosage of gadolinium-based contrast agents (GBCAs) while maintaining accurate detection and enhancement of BBB openings. A spatiotemporal network (ST-Net) is introduced, combining spatial and temporal networks, allowing for the extraction of diagnostic quality images with reduced GBCAs dosage.

### CROSS-CONFERENCE INFORMATION

This application is a continuation of International PCT Application No. PCT/US2024/015775, filed on Feb. 14, 2024, which claims the priority of U.S. Provisional Application Ser. No. 63/446,163, filed on Feb. 16, 2023, the entire contents of which are incorporated by reference herein.

### STATEMENT REGARDING FEDERALLY SPONSORED RESEARCH

This invention was invention was made with government support under Matheson Foundation Grant No. UR010590 and Herbert Irving Cancer Center Support Grant No. P30CA013696. The government has certain rights in the invention.

### BACKGROUND

The disclosed subject matter relates to the field of medical imaging, specifically to the detection and enhancement of blood-brain barrier (BBB) openings using deep learning techniques.

Techniques to open the BBB can be used to allow substances to enter the central nervous system (CNS), optimizing drug delivery. A BBB opening can be detected using Magnetic Resonance Imaging (MRI) with gadolinium-based contrast agents (GBCAs).

However, repeated use of GBCAs can lead to accumulation and be retained in body tissues, including the brain. In addition, the contrast-based sequences can extend MRI scanning time, leading to increased costs, patient discomfort, and movement/motion artifacts.

As such, there is a need in the art for a technique for providing an alternative detection with a reduction or substitution of GBCAs dosage.

### SUMMARY

The disclosed subject matter provides methods and system employing deep learning to address the challenge of reducing the dosage of gadolinium-based contrast agents (GBCAs) while maintaining accurate detection in medical imaging.

An exemplary method for reducing dosage of GBCAs in medical imaging includes applying dynamic contrast-enhanced magnetic resonance imaging (DCE-MRI) to a subject to obtain a plurality of DCE-MRI images; analyzing the plurality of DCE-MRI images with a deep learning model using a spatiotemporal network to obtain a corresponding plurality of Volume Transfer Constants (Kₜᵣₐₙₛ); and forming a Kₜᵣₐₙₛ map using the plurality of Kₜᵣₐₙₛ.

In certain embodiments, analyzing the plurality of DCE-MRI images includes extracting spatial information from the scans using a three-dimensional convolutional neural network (CNN) encoder. In certain embodiments, analyzing the plurality of DCE-MRI images also includes concatenating the spatial information with two reference arrays, including average intensity of pre-contrast images and average DCE-MRI time series signal. In certain embodiments, analyzing the plurality of DCE-MRI images further includes implementing a temporal network, including a one-dimensional CNN layer to blend spatial and reference information, and two separate CNN pathways capturing long-term and short-term temporal characteristics. In certain embodiments, analyzing the plurality of DCE-MRI images further includes fusing long-term and short-term temporal characteristics for outputting, using additional one-dimensional CNN layers and a fully connected layer.

In certain embodiments, the deep learning model is trained on a dataset including employing BBB-opening patches.

In certain embodiments, applying DCE-MRI includes inducing FUS with administration of microbubbles to BBB-openings.

In certain embodiments, the analyzing the plurality of DCE-MRI images includes processing spatial and temporal information simultaneously by treating 3D input as a single entity for a CNN encoder. The analyzing can include processing 3D patches of the DCE-MRI data and applying linear embedding followed by the CNN encoder to capture spatiotemporal features.

In certain embodiments, the analyzing the plurality of DCE-MRI images comprises extracting local spatial information from input patches, followed by a CNN encoder to capture global spatiotemporal relationships.

In certain embodiments, the method further includes injecting contrast agents to trace the BBB-opening. In certain embodiments, contrast agents are injected at two times. In certain embodiments, contrast agents are injected first with low dose, and then injected with full dose.

In certain embodiments, the Kₜᵣₐₙₛ map is formed through a general kinetic model (GKM) model. In certain embodiments, employing BBB-opening patches includes cropping each voxel of whole brain (WB) scan into patches for extracting spatial information.

Furthermore, an exemplary medical imaging system integrating deep learning includes a dynamic contrast-enhanced magnetic resonance imaging (DCE-MRI) apparatus configured to obtain a plurality of DCE-MRI images; and a processing unit configured to implement one or more exemplary techniques to analyze the plurality of DCE-MRI images to output a corresponding plurality of Kₜᵣₐₙₛ.

In certain embodiments, the medical imaging system further includes a display unit configured to present a Kₜᵣₐₙₛ map on visual representations of the plurality of Kₜᵣₐₙₛ. In certain embodiments, the DCE-MRI apparatus is further configured to adjust imaging parameters based on the plurality of Kₜᵣₐₙₛ. In certain embodiments, the processing unit is further configured to store the plurality of Kₜᵣₐₙₛ in a storage device for subsequent analysis. In certain embodiments, a FUS apparatus is further integrated to the DCE-MRI apparatus for inducing BBB-opening.

### BRIEF DESCRIPTION OF THE DRAWINGS

The accompanying drawings, which are incorporated and constitute part of this disclosure, illustrate some embodiments of the disclosed subject matter.

FIG. 1A illustrates a procedure of a deep learning process for DEC-MRI scans with GBCAs according to some embodiments of the disclosed subject matter. FIG. 1B illustrates a timeline for the procedure in FIG. 1A. FIG. 1C illustrates an image preprocessing pipeline in FIG. 1A.

FIG. 2 illustrates a ST-Net architecture for Convolutional Neural Network (ST-CNN) according to some embodiments of the disclosed subject matter.

FIG. 3 illustrates a comparison on Full dose and low dose volume transfer constant (Kₜᵣₐₙₛ) map and the residual map from different model according to some embodiments of the disclosed subject matter.

FIG. 4 illustrates a Kₜᵣₐₙₛ map in a three-dimensional brain volume from different model according to an embodiment of the disclosed subject matter. FIG. 5 illustrates a box plot visualizing model performance across ten testing subjects according to some embodiments of the disclosed subject matter.

FIG. 6 illustrates an exemplary medical imaging system according to some embodiments of the disclosed subject matter.

FIG. 7 illustrates a Spatiotemporal 3D Vision Transformer (ST-VIT) architecture according to some embodiments of the disclosed subject matter.

FIG. 8 illustrates a Spatial-then-Spatiotemporal 3D Vision Transformer (SST-ViT) architecture according to some embodiments of the disclosed subject matter.

FIG. 9A-9F illustrates training and validation loss curves for the three models according to some embodiments of the disclosed subject matter.

FIG. 10 illustrates reconstructed Kₜᵣₐₙₛ maps and the residual maps for various models according to some embodiments of the disclosed subject matter.

Throughout the figures and specification, the same reference numerals are used to indicate similar features and/or structures.

### DETAILED DESCRIPTION

The disclosed subject matter discloses a deep learning technique for analyzing and reducing contrast agent dosage in MRI scans in medical application, e.g., modeling BBB openings. The disclosed subject matter not only creates images indicating the transfer of substances (Kₜᵣₐₙₛ) with reduced contrast agent, but also provides enhanced quality MRI scans with lower contrast agent doses, through a Spatiotemporal Network (ST-Net).

### Definitions

Herein, the term “Blood-Brain Barrier”, short for BBB, refers to a boundary that separates the blood circulating in the body from the brain's extracellular fluid. Typically, this barrier is crucial for maintaining the brain's microenvironment and protecting it from harmful substances. The selective nature of the BBB causes challenges in delivering therapeutic drugs to the brain. Many drugs have difficulty crossing this barrier, limiting their effectiveness in treating neurological conditions. The term “Blood-Brain Barrier opening”, short for “BBB opening” or “BBB-opening”, refers to a temporary disruption or alteration in the integrity of the BBB. Under specific conditions, the BBB can be manipulated to become more permeable, allowing substances that would normally be restricted to enter the brain tissue more freely, especially for the purpose of facilitating the delivery of drugs or therapeutic agents to the brain.

Herein, the term “Focused Ultrasound”, short for FUS, refers to a technique involving directing ultrasound waves precisely to a specific area of the brain. The energy from the ultrasound can transiently disrupt the BBB, creating temporary openings that allow for the delivery of therapeutic agents.

Herein, the term “Gadolinium-Based Contrast Agents”, short for GBCAs, refers to substances commonly used in medical imaging procedures such as magnetic resonance imaging (MRI). These agents contain gadolinium, a paramagnetic metal, which enhances the visibility of internal body structures in imaging by altering the magnetic properties of surrounding water molecules. GBCAs are administered intravenously and help improve the diagnostic accuracy of MRI scans, particularly in visualizing organs, blood vessels, and abnormalities. However, concerns have been raised about the retention of gadolinium in the body, leading to potential long-term health effects, and research is ongoing to address these safety considerations.

Herein, the term “Dynamic Contrast-Enhanced Magnetic Resonance Imaging”, short for DCE-MRI refers to a medical imaging technique used to assess the perfusion and vascularity of tissues by tracking the passage of a contrast agent through blood vessels.

Herein, the term “Convolutional Neural Networks”, short for CNNs, refers to a class of artificial neural networks specifically designed for processing structured grid data, such as images. CNNs are widely used in various fields, particularly in computer vision tasks, due to their effectiveness in capturing spatial hierarchies and patterns within data. Typically, a three-dimensional convolutional neural network (CNN) encoder can be utilized to extract spatial information from the DCE-MRI scans.

Herein, the term “subject” refers to an individual or entity participating in a medical study, experiment, clinical trial, or any form of research investigation. Subjects can be diverse and may include: human, animals, or cellular.

Herein, the term “spatiotemporal network”, short for ST-Net in deep learning models or approaches, refers to a specific neural network architecture utilized in medical imaging, particularly in the context of DCE-MRI. ST-Net is designed for analyzing DCE-MRI scans, which incorporates both spatial and temporal information to predict key parameters related to contrast agent dynamics in medical imaging, capable to capture changes in contrast over time, particularly focusing on perfusion and vascular properties in tissues. The term “Temporal Network” refers to a type of network designed to analyze and extract information from data that changes over time. Herein, Temporal Network is part of the overall ST-Net architecture used for analyzing DCE-MRI scans. A Spatial Network is designed to analyze and extract information from the spatial characteristics of data, particularly when dealing with images or multidimensional datasets. In certain embodiments, the disclosed subject matter provides at least three deep learning approaches for efficient Kₜᵣₐₙₛ map reconstruction: spatiotemporal 3D Convolutional Neural Network (ST-CNN), Spatiotemporal 3D Vision Transformer (ST-ViT), and a hybrid Spatial-then-Spatiotemporal 3D Vision Transformer (SST-ViT).

Herein, the term “Volume Transfer Constant, short for “Kₜᵣₐₙₛ” or “Kₜᵣₐₙₛ value”, referred to a perfusion parameter value of embodying the vascular transfer coefficient and reflecting vascular permeability. It denotes the transfer rate at which contrast agent (such as GBCAs) moves from the blood plasma to the extravascular extracellular space of each voxel, modeling the capillary permeability and hence can be used to detect BBB-opening. The measurement of Kₜᵣₐₙₛ value is typically done through DCE-MRI, where a series of images is acquired before, during, and after the injection of a contrast agent. The changes in signal intensity over time are used to estimate Kₜᵣₐₙₛ. The term “Kₜᵣₐₙₛ map” refers to a visual representation of the Kₜᵣₐₙₛ, derived from DCE-MRI. The Kₜᵣₐₙₛ map provides information about the transfer rate of contrast agent from the bloodstream to the tissue and is particularly useful in assessing vascular properties and permeability in various organs or tissues.

### EXAMPLES

In the section below, the examples or embodiments herein are provided merely for descriptive purposes, rather than being restrictive or limiting to the disclosed subject matter.

The methods and systems provided in the disclosed subject matter herein are useful for opening a tissue utilizing microbubbles and focused ultrasound at certain acoustic parameters. Although the description provides some examples opening the BBB, the methods and systems herein can be applied for opening other tissues, such as muscular tissue, liver tissue or tumorous tissue, among others. In particular, the disclosed subject matter provides three efficient deep learning model examples for Kₜᵣₐₙₛ reconstructions: spatiotemporal 3D Convolutional Neural Network (ST-CNN), Spatiotemporal 3D Vision Transformer (ST-ViT), and a hybrid Spatial-then-Spatiotemporal 3D Vision Transformer (SST-ViT). Examples have demonstrated that these models can achieve superior performance, with significant improvements compared to the conventional models for full dose or low dose DCEMRI scans. The proposal architectures in the disclosed subject matter provide an more accurate Kₜᵣₐₙₛ map reconstruction that takes only a fraction of the time compared to conventional methods by extracting spatiotemporal information rapidly and accurately in a single pass.

### Procedure

An animal model was implemented. In certain embodiments, nine C576 J/BL mice at the age of 3-6 months are scanned using the DCE-MRI protocol described later. A total of 162 scans were acquired for nine subjects.

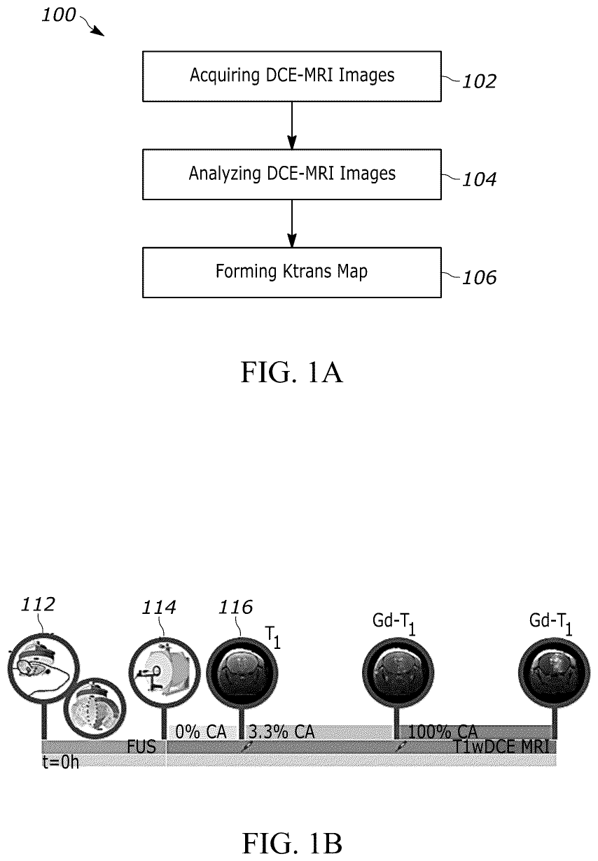

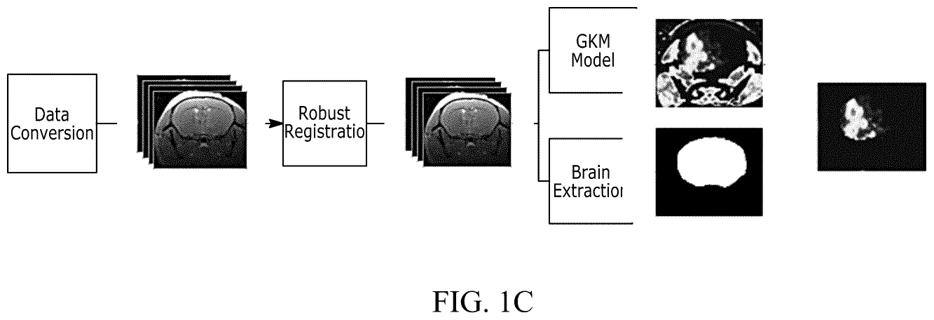

As shown in FIG. 1A, an exemplary procedure for the disclosed subject matter can include,
acquiring dynamic contrast-enhanced magnetic resonance imaging (DCE-MRI) images (102);
analyzing the DCE-MRI images (104); and
forming a Kₜᵣₐₙₛ map (106).

In some exemplary embodiments, at 102, the MRI apparatus is configured to capture dynamic contrast-enhanced DCE-MCI scans images, utilizing contrast agents to enhance the visibility of blood flow and tissue characteristics, where the FIS-induced BBB-opening is typically detected by applying MRI to a subject. Subsequently, at 104, the acquired raw DCE-MRI images are subjected to analysis by employing a deep learning model proposed by the subject matter, specifically utilizing a spatiotemporal network (ST-Net), which is composed of a spatiotemporal convolutional neural network (CNN)-based deep learning architecture with a three-dimensional CNN encoder, to improve the deep learning performance. Additionally, the deep learning model is trained to recognize complex patterns and temporal variations within the dynamic contrast-enhanced images, enabling the extraction of comprehensive information related to tissue characteristics, blood perfusion, and spatial-temporal dynamics. As a result of the above analysis at 104 performed by the spatiotemporal network, a plurality of Kₜᵣₐₙₛ corresponding to a plurality of DCE-MRI images is generated. Following, at 106 the plurality of Kₜᵣₐₙₛ is reconstructed to form a Kₜᵣₐₙₛ map, depicting a whole brain information visually.

It is to be understood that these procedures represent an exemplary embodiment, and variations within the scope of the disclosed subject matter are contemplated. The utilization of deep learning models, analysis of the imaging scans using a ST-Net and the extraction of the Kₜᵣₐₙₛ value contribute to an advanced methodology for characterizing tissue properties and dynamics.

FIG. 1B illustrates a timeline for different procedures of the exemplary method in FIG. 1A. In certain embodiments, prior to performing imaging scans, a FUS 112 disrupts a tissue of a subject, e.g., BBB, by injecting microbubbles to induce openings. After a substantial duration of FUS application, e.g., twelve hours, the BBB opening can be stable, and then the subject is placed on an MRI apparatus 114 and scanned for the baseline for the first four acquisitions on imaging data 116. In certain embodiments, contrast agents can be injected at multiple times with different dosage instructions. For example, Ten mmol/kg contrast agent (3.3% of the full dosage, low dose) Gadodiamide is first injected, and consequently, eighty T1−weighted (T1−W) DCE-MRI images can be acquired. Following the injection of the remaining 97.7% contrast agent (full dose), eighty-four T1−W DCE-MRI can be acquired. The contrast agents after the above two injections trace BBB openings.

In certain embodiments, regarding the FUS-induced BBB-opening procedure, FUS can be applied with microbubbles. In particular, a single-element spherical-segment FUS transducer, driven by a function generator and power amplifier, is used for sonication. In-house microbubbles (8×108 bubbles/mL, diameter: 1.37±1.02 μm) are intravenously injected after dilution in saline solution to 200 μL. Sonication parameters include 0.5 MHz frequency, 0.3 MPa peak-negative pressure, and 10 ms bursts at 5 Hz repetition over 120 s (600 pulses).

Following FUS, the imaging scans are performed. In certain embodiments, the subject is scanned using a Bruker BioSpec94/20 scanner (9.4 T) with Para Vision 6.0.1 software. Anesthesia (3% isoflurane for induction, 1.1-1.5% for maintenance) is administered at 1 liter/min via a nose cone. DCE-MRI employs a 2-D FLASH T1-weighted sequence (180×150×18×84 matrix, spatial resolution 100×100 μm², 500 μm slice thickness, TR/TE=200/2.12 ms) before and during intraperitoneal injections of Gadodiamide (Gd).

Upon these above procedures, the raw imaging data including four-dimensional DCE T1-weighted brain (WB) MRI of 18 slices and 84 acquisitions, with a total acquisition time of about 1 hour, can be acquired.

The acquired imaging data can be further preprocessed for facilitating imaging data analysis. In certain embodiments, the plurality of DCE-MRI images are converted to NIFTI format and underwent within-subject robust registration using software, e.g., FreeSurfer. Additionally, a WB mask can be manually labeled in 3DSlicer for model training. FIG. 1C illustrates an exemplary raw image preprocessing pipeline. As shown in FIG. 1C, the plurality of DCE-MRI images from DICOM format to Neuroimaging Informatics Technology Initiative (NIFTI) format 118 are first converted, and within-subject robust registration is performed. Following above, a deep learning proposed by the disclosed subject matter is used to analyze the plurality of DCE-MRI images to output a corresponding plurality of Kₜᵣₐₙₛ. Then, a Whole Brain (WB) Kₜᵣₐₙₛ map 120 can be generated by reconstructing the plurality of Kₜᵣₐₙₛ through the general kinetic model (GKM) model. Finally, the WB Kₜᵣₐₙₛ map 120 with the manually labeled brain mask are extracted. To facilitate model training, the WB mask is manually labeled in 3DSlicer. Refer to FIG. 1C for an illustration of the exemplary raw image preprocessing pipeline. Selectively, the WB Kₜᵣₐₙₛ map 120 is extracted, incorporating the manually labeled brain mask for precise delineation. This comprehensive preprocessing pipeline ensures that the acquired imaging data is appropriately formatted and aligned, laying the groundwork for subsequent detailed analysis and extraction of valuable insights, such as the WB Kₜᵣₐₙₛ map, through certain modeling techniques.

At the procedure of employing the deep learning model, the various models can be developed to predict full-dose Gd BBB-opening from full-dose and/or low-dose Gd DCE-MRI. The deep learning network employs a patch-based strategy to predict Kₜᵣₐₙₛ values. After Kₜᵣₐₙₛ reconstruction, a Kₜᵣₐₙₛ map (Estimation) is generated and matched with the Ground Truth. The deep learning network includes multiple parts separately, simultaneously, or hybrid, which are illustrated in the FIGs and detailed below.

### ST-CNN Model and Results

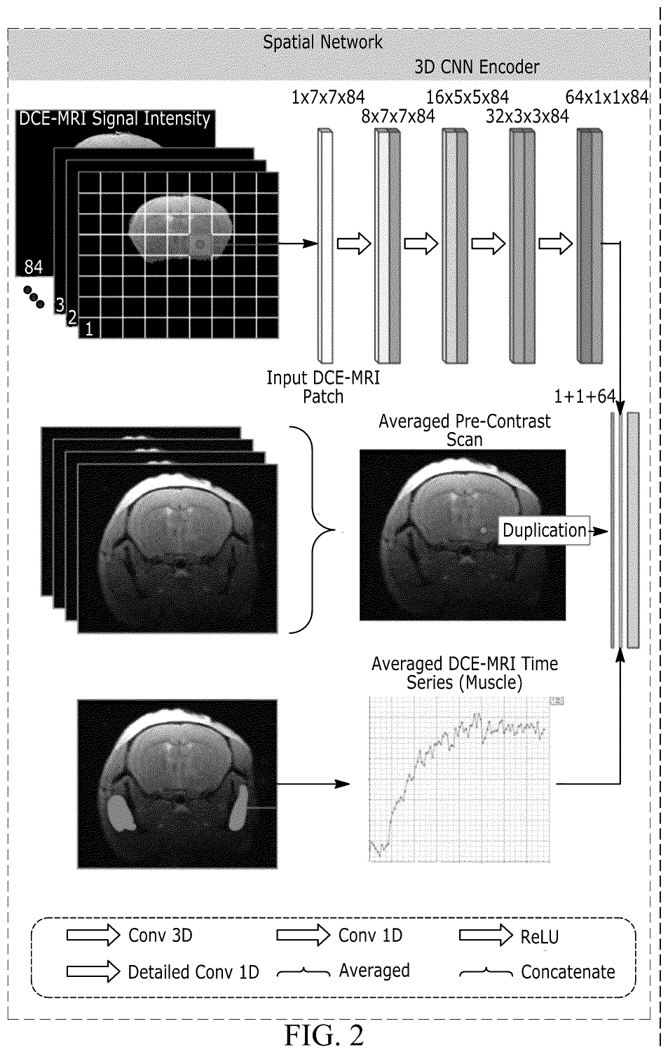

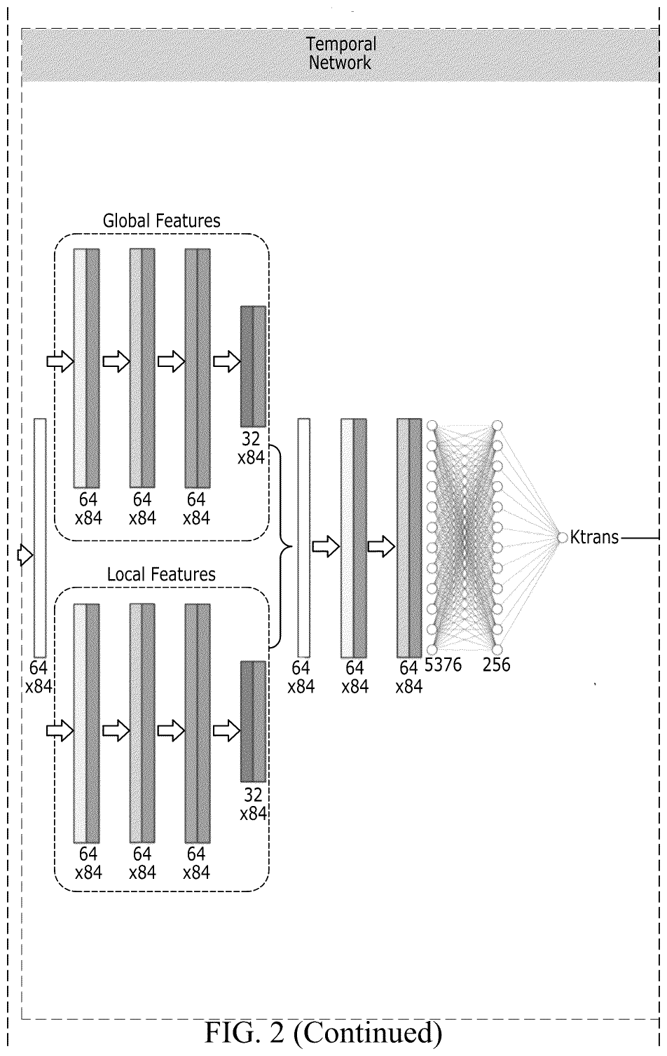

With reference to FIG. 2, an exemplary ST-Net architecture 200 for the ST-CNN model is illustrated. In certain embodiments, the DCE-MRI scans image acquired by the MRI apparatus are analyzed as follows.

In a general work frame, the ST-CNN model first extracts spatial feature (network) through the 3D CNN encoder, which then extract temporal features (network) through a 1D CNN layer and a dilated CNN layer. For temporal features, two reference inputs are used: (1) the average intensity at the patch center from pre-contrast MRI scans, and (2) the averaged time-series signal from surrounding muscle tissue in the DCE-MRI data, obtained through segmentation-based muscle masking.

The acquired DCE-MRI images are first cropped to patches with a specific size, e.g., 7×7×84 input DCE-MRI patches 210, and spatial information are extracted by a three-dimensional convolutional neural network (CNN) encoder 220. This model utilizes ten whole-brain DCE-MRI scans from mice with FUS-induced BBB opening, processed into overlapping patches with a cropped dimension, e.g., 7×7×84 (x, y, time) based on the dataset of the DCE-MRI data. To balance the training dataset, patches with zero Kₜᵣₐₙₛ values were randomly dropped, while full scans were used for testing. Ground-truth Kₜᵣₐₙₛ maps are derived using the Tofts model with low dose and/or full-dose contrast, depending on the eligibility of various models. Models were implemented in PyTorch and trained with the Adaptive Moment Estimation (Adam) optimizer (learning rate 5e-4, batch size 256) using MAE loss, five-fold cross-validation, early stopping, and dropout.

The training for models was selected to various training time duration depending on the various scan contrast dosages, e.g., selectively but not limited to, ˜1 hour for full-dose scans and ˜90 minutes for low-dose scans. More complex models (e.g., ST-CNN and SST-ViT) were trained for ˜90 minutes to ensure convergence. Performance was evaluated using the following metrics: Spatial Cross-Correlation (SCC), Pearson Correlation Coefficient (PCC), Concordance Correlation Coefficient (CCC), Normalized Root Mean Square Error (NRMSE), Kullback-Leibler divergence (KL), Peak Signal-to-Noise Ratio (PSNR), and Structural Similarity Index Measure (SSIM), with statistical significance assessed via t-tests (p<0.05).

Following with a spatial feature extraction, the spatial information is concatenated with two reference arrays 230, 240: (1) average intensity of the four pre-contrast images at the center of the patch(es) and (2) the average DCE-MRI time series signal from Muscle (e.g., muscular tissue). The first reference array 230 represents the average intensity of the four pre-contrast images at the center of the patch(es), enhancing the model's understanding of baseline characteristics. The second reference array 240 integrates the average DCE-MRI time series signal from Muscle (e.g., muscular tissue), providing additional context to the spatial information. This concatenation process ensures that essential details regarding both spatial and reference information are effectively captured.

Thereby, the essential details about the spatial information of the MRI images can be captured. The three concatenated inputs are fed into the Temporal network subsequently. A one-dimensional CNN layer is used to combine spatial and reference information, extracting fundamental temporal features. Then, two separate CNN pathways are employed to capture long-term (global feature 212) and short-term (local feature 214) temporal characteristics. Finally, these long-term and short-term details are fused using two more one-dimensional CNN layers, and a fully connected layer to predict the full dose Kₜᵣₐₙₛ value 216 for the center point of each patch.

In certain embodiments, a Leaky Rectified Linear Unit (ReLU) activation can follow each fully connected layer, enhancing the model's capacity to capture complex relationships in the data. The size of the output features for each layer is thoughtfully provided in FIG. 2. The resulting Kₜᵣₐₙₛ values 216 are then reconstructed to generate a Kₜᵣₐₙₛ map (Estimation) 218, which is compared against the Ground Truth 222 for validation. This meticulous architecture ensures that the ST-Net architecture 200 effectively integrates spatial and temporal information for accurate prediction of Kₜᵣₐₙₛ values, contributing to the reliable estimation of the underlying dynamics in the DCE-MRI data.

Regarding the spatial network in certain embodiments, each voxel from the entire WB scan is resized into 7×7×84-sized patches. A 3D convolutional neural network (CNN) encoder is then applied to extract and preserve spatial features. The final output featured Kₜᵣₐₙₛ map from this encoder network is of dimension 64×1×1×84. Subsequently, this featured map is compressed into a 64×84 vector. The introduce of a patch step rate can reduce the overlapping area between input patches.

Illustrated in FIG. 2, following the spatial network processing, the output from the encoder network (64×84) is integrated with two reference inputs. The first reference input calculates the average intensity at the patch center from the initial four acquisitions before contrast agent administration, broadcasting this value 84 times (1×84) to match the encoder's output dimensions. The second reference input is a 1×84 averaged time-series signal from DCE-MRI data in surrounding Muscles. These three inputs form a concatenated array (66×84) fed into a Temporal Network inspired by the fast-eTofts model. The temporal model uses one-dimensional CNN layers to fuse spatial and reference data, extract low-level temporal features, and employ parallel global and local pathways for long-term and short-term temporal features. Finally, two one-dimensional CNN layers and a fully connected layer predicted the full dose Kₜᵣₐₙₛ value for each patch's center point. Dropout layers are included to prevent overfitting. Predicted Kₜᵣₐₙₛ values are used to reconstruct a Kₜᵣₐₙₛ map in certain embodiments.

In certain embodiments, the ST-Net can be further trained and refined using an optimizer with a mean absolute error (MAE) loss function and early stopping at 300 epochs. The 3D CNN encoder in the spatial network consists of 4 Convolutional Layers with 3×3×1 kernels, starting with 1 input channel and ending with 64 output channels (as shown in FIG. 2). The 1D Convolutions in the Temporal Network also use a 3-sized kernel. To fine-tune the deep learning model, the ST-Net can be trained with a batch size of 512, a learning rate of 1e-4, and the addition of four CNN encoder layers without batch normalization. Training utilized three 24 GB NVIDIA Quadro 6000 GPUs with PyTorch.

In certain embodiments, the dataset details for the deep learning model can be selected flexibly. For example, regarding the dataset selection, two subjects are repeatedly chosen for testing, while the remaining eight are used for training. The WB voxel data of the eight subjects are shuffled and split into a four-to-one ratio for training and validation. Pursuant to the abnormal value removal in certain embodiments, no filters are applied to the input DCE-MRI and ground truth Kₜᵣₐₙₛ map. All input data DCE-MRI patches averaged pre-contrast scan, and averaged Gd concentration in muscle) are normalized by the 99th percentile of the averaged pre-contrast scan. To mitigate the impact of noise-induced extreme values in the Kₜᵣₐₙₛ maps, only voxels with Kₜᵣₐₙₛ values in the range of [0, 0.05] l/min are considered when calculating the loss.

Brain and BBB-opening regions are manually outlined using 3DSlicer. Two sets of ROIs (regions of interest) are selected for each mouse dataset, one covering all BBB-opening voxels and the other from normal-appearing brain tissue with a four-fold greater voxel count.

To evaluate the quality of the predicted K-trans map, similarity between the predicted K-trans map generated by deep learning and the ground truth K-trans map derived using experimental data from the DCE protocol is analyzed. To investigate any advantage of adding a spatial network, the comparison between ST-Net and the modified fast-eTofts, a purely Temporal network (T-Net) is displayed visually. Additionally, an evaluation on the GKM derived low dose image to show the improvement of detecting BBB-opening using deep learning is performed.

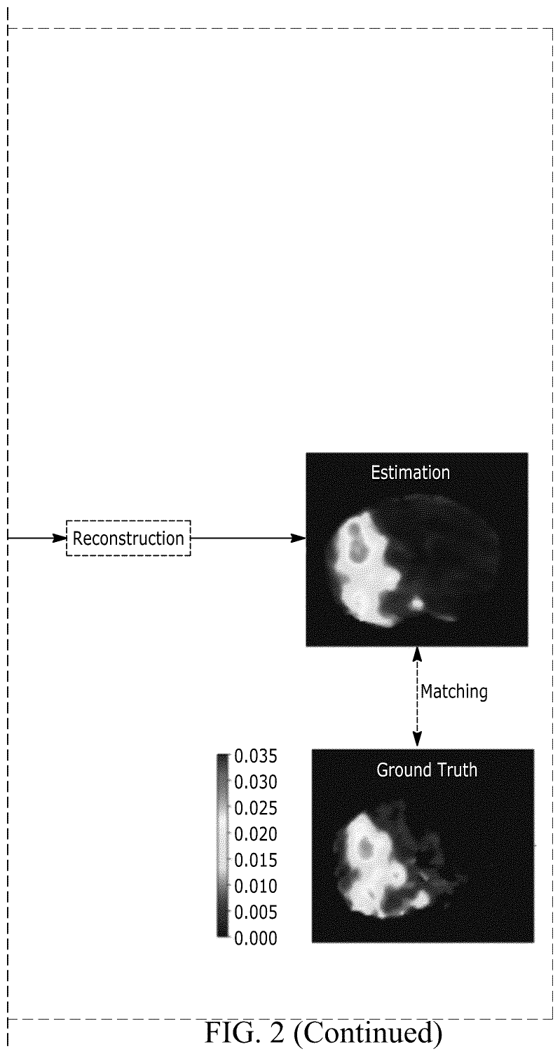

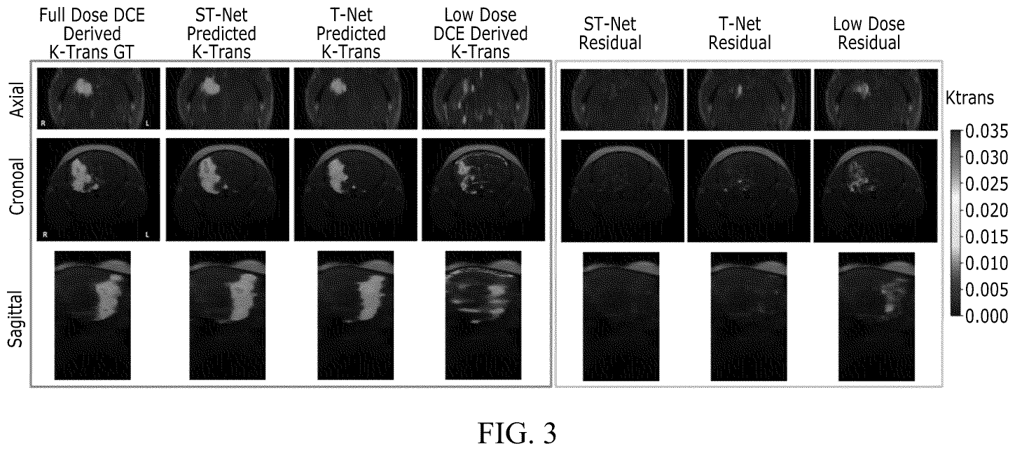

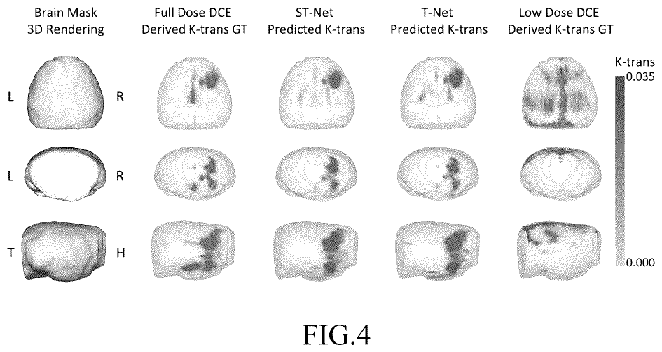

The performance between the proposed ST-Net with low contrast agent dosage Kₜᵣₐₙₛ images derived from the conventional GKM model and the Temporal-only deep learning model, T-Net, are compared. The derived/predicted 2D Kₜᵣₐₙₛ images for one testing subject from three different orientations are visualized in FIG. 3. As shown in FIG. 3, the first column is the full dose Kₜᵣₐₙₛ images derived by conventional GKM fitting and is used as ground truth in the deep learning model. The second to fourth columns are low-dose Kₜᵣₐₙₛ images mapped by the GKM model, predictions by T-Net, and predictions by ST-Net, respectively. The following four columns display the residual differences between full dose and derived/predicted Kₜᵣₐₙₛ images. Additionally, three-dimensional renderings of BBB-opening for low dose, full dose, T-Net, and ST-Net are shown in FIG. 4. An “iron” color scheme is applied in FIG. 4. (L: left; R: right; T: tail; H: head)

In certain embodiments, noise from the GKM derived and deep learning predicted Kₜᵣₐₙₛ images is first removed using a 3D median filter with local window-size 3×3×3 from Python library-SciPy. The post-processed WB Kₜᵣₐₙₛ maps are then used to visualize and quantify the performance of the algorithms mentioned above (ST-Net, T-Net, GKM derived low dose images) using structural similarity index (SSIM) (1), peak signal-to-noise ratio (PSNR) (2), Pearson correlation coefficient (PCC) (3), concordance correlation coefficient (CCC) area under the curve (AUC), and normalized root mean square error (NRMSE) (4) metrics. Significant differences are illustrated in box plots, with asterisks denoting the level of significance (*: p<0.05; **: p<0.01; ***: p<0.001; ****: p<0.0001), illustrated in FIG. 4. The metrics can be calculated as follows:

$$SSIM = {l}^{a} ( x , y ) {c}^{β} ( x , y ) {s}^{γ} ( x , y ) \qquad ( 1 )$$

$$PSNR = 10 \cdot {log}_{10} ( \frac{{MAX}_{x}^{2}}{MSE} ) \qquad ( 2 )$$

$$PCC = \frac{{σ}_{xy}}{{σ}_{x} {σ}_{y}} \qquad ( 3 )$$

$$CCC = \frac{2 {σ}_{xy}}{{( {μ}_{x} - {μ}_{y} )}^{2} + {σ}_{x}^{2} + {σ}_{y}^{2}} \qquad ( 4 )$$

$$NRMSE = \frac{\sqrt{\frac{1}{N} \sum {( x - y )}^{2}}}{\sqrt{\frac{1}{N} \sum {x}^{2}}} \qquad ( 5 )$$

Where x and y represent the voxel of ground truth and derived/predicted images. The l(x, y), c(x, y), and s(x, y) in SSIM respectively measure the differences between the luminance, contrast, and structure of the two images, and α, β, and γ are three constants. MAXₓ and MSE in PSNR represent the maximum voxel intensity of the ground truth and the mean square error of the two images. μₓ and μₓ are the means for the two images, and σₓ and σ_{y} are the corresponding variances.

σ_{xy} is the covariance and N is the voxel number within the ROI. Both ST-Net and T-Net networks are trained using five-fold cross-validation and the t-test is performed on the metrics mentioned above. Significant differences are shown in box plots, with each number of * indicating the order of significance (*: p<0.05; **: p<0.01, ***: p<0.001, *: p<0.0001).

The quantitative comparison among low dose, T-Net, and ST-Net on all the ten testing subjects for WB and opening area, are summarized in Table 1.

TABLE 1

| Metrics | | | |
|---|---|---|---|
| (Averaged) | ST | Temporal | Low-Dose |
| SSIM↑ | 0.978 ± 0.028 | 0.984 ± 0.016 | 0.934 ± 0.019 |
| PSNR↑ | 22.772 ± 3.745 | 22.876 ± 2.582 | 12.916 ± 3.602 |
| PCC↑ | 0.759 ± 0.075 | 0.714 ± 0.120 | 0.162 ± 0.127 |
| CCC↑ | 0.663 ± 0.174 | 0.573 ± 0.227 | 0.118 ± 0.118 |
| AUC↑ | 0.775 ± 0.091 | 0.745 ± 0.067 | 0.596 ± 0.046 |
| NRMSE↓ | 0.698 ± 0.218 | 0.685 ± 0.159 | 2.310 ± 1.069 |

The average performance with standard deviation is shown in the last row of Table 1. In the post-processed reconstructed WB Kₜᵣₐₙₛ maps, ST-Net achieved the highest PCC (0.759+0.075), CCC (0.663+0.174), and AUC (0.775+0.091) across ten testing mice. On the other hand, T-Net performed better in SSIM (0.984+0.016), PSNR (22.876+2.582), and NEMSE (0.685+0.159). Table 2 shows a quantitative Kₜᵣₐₙₛ map comparison obtained from the general kinetic model (GKM) method and deep learning algorithm for the BBB-opening region. As shown in Table 2, for the BBB-opening area, the ST-Net model outperformed in every metrics (SSIM=0.959+0.029, PSNR=28.790+4.366, PCC=0.799+0.055, CCC=0.697+0.187, AUC=0.803+0.040, and NRMSE=0.456+0.175). The box plots across the ten testing subjects for each metric in two ROIs are shown in FIG. 5. FIG. 5 also shows that both T-Net and ST-Net have significant differences compared to low dose Kₜᵣₐₙₛ images in both WB and BBB-opening only areas. ST-Net and T-Net also show significant differences in the opening areas for every metric.

TABLE 2

| Metrics | | | |
|---|---|---|---|
| (Averaged) | ST | Temporal | Low-Dose |
| SSIM↑ | 0.959 ± 0.029 | 0.940 ± 0.048 | 0.911 ± 0.019 |
| PSNR↑ | 28.790 ± 4.366 | 25.747 ± 4.025 | 22.712 ± 5.016 |
| PCC↑ | 0.799 ± 0.055 | 0.715 ± 0.129 | 0.358 ± 0.122 |
| CCC↑ | 0.697 ± 0.187 | 0.528 ± 0.256 | 0.265 ± 0.130 |
| AUC↑ | 0.803 ± 0.040 | 0.773 ± 0.054 | 0.690 ± 0.068 |
| NRMSE↓ | 0.456 ± 0.175 | 0.575 ± 0.200 | 0.985 ± 0.474 |

In the above embodiments, the disclosed subject matter has provided an effective deep learning method featured with a ST-Net, a spatiotemporal CNN deep learning architecture designed for predicting a full dose time-series BBB-opening by low dose T1W MRI. From the experimental results, the disclosure has not only successfully investigated the efficacy of detecting BBB-opening with low dosage contrast agent administration but also improved the model performance with an additional 3D CNN.

The above embodiments of the disclosed subject matter have validated a deep learning algorithm that can be used to acquire full dose Kₜᵣₐₙₛ maps while decreasing contrast agent dosage using T-Net. The comparison from three directions in FIG. 3 shows a high similarity between T-Net and ground truth. Compared to low dose derived K-trans, T-Net depicts the BBB-opening area and outlines more accurately. However, it has been noted that the edges of the BBB-opening in T-Net look noisy in the residual maps, which is caused by the model's deficiency on differentiating the boundaries between opening and non-opening tissues. The intensity observed within the BBB-opening being lower than the ground truth is caused by the same above reason. The intensity of the FUS focus point of the BBB-opening in the Kₜᵣₐₙₛ map is expected to be the highest. However, since T-Net only learned the contrast agent concentration changes for each pixel, it cannot detect the intensity difference among adjacent pixels.

As a solution, the disclosed subject matter provides adding a spatial network to share the features across the brain, which has been proven to further enhance the performance of predicting Kₜᵣₐₙₛ while retaining high fidelity. Instead of simply inputting data on the voxel level, the disclosed subject matter crops the WB ROI to patches across time and extracts the spatial features for each patch through a three-dimensional CNN encoder. With spatial information, the proposed ST-Net is able to learn the brain structure, and predict the BBB-opening location and shape in reference to the neighbor voxels. The results in FIG. 5 show there are significant differences between ST-Net predicted results and GKM-derived low dose BBB-opening among all metrics in both ROIs. The 2D whole-head (WH) Kₜᵣₐₙₛ images overlapped with structural MRI scans in FIG. 3 and the 3D WB volumes in FIG. 4 visualize one of the testing subjects. Both FIGs show a clear BBB-opening in ST-Net; however, the opening in the low dose image is barely visualized. The results demonstrate the efficiency and potential of using ST-Net with a low-dose contrast agent in detecting BBB-opening.

The improvements of adding a spatial network to ST-Net include increasing model robustness and improving the prediction at the edges of the BBB-opening. The box plot in FIG. 5 shows the SSIM, PSNR, CCC, and SSIM significantly increase and the NRMSE significantly decreases within the opening area in ST-Net. The standard deviations of ST-Net for all metrics for both ROIs are the smallest, demonstrating that the spatial network is a crucial element in predicting 3D images by providing spatial information from neighbor voxels. The PSNR of the ST-Net opening area is significantly improved in FIG. 5 shows ST-Net provides a denoise effect. The statistical improvement can be visualized in the sagittal direction in FIG. 3. The comparison shows that ST-Net is predicted better on the opening boundary, and the opening edge is less noisy than T-Net model. Moreover, the intensity of the BBB-opening in ST-Net is observed to be more similar to the ground truth and matches the structure of the BBB-opening. As a result, adding a spatial network proved to be of high value/importance.

The results show that ST-Net not only predicts the BBB-opening area accurately, but the normal-appearing brain tissues also show a high resemblance to the ground truth in ST-Net. ST-Net effectively modeled both BBB-opening and non-opening areas. Selectively, FUS parameters, sample populations, and multi-modality MRI sequences can be adjusted to eliminate the need for contrast agents. The disclosed subject matter has been proved to be valid in reducing the use of GBCAs and the risk of contrast agent-induced side effects, thereby improving the safety profile of FUS treatments in the brain.

### ST-VIT Model and Results

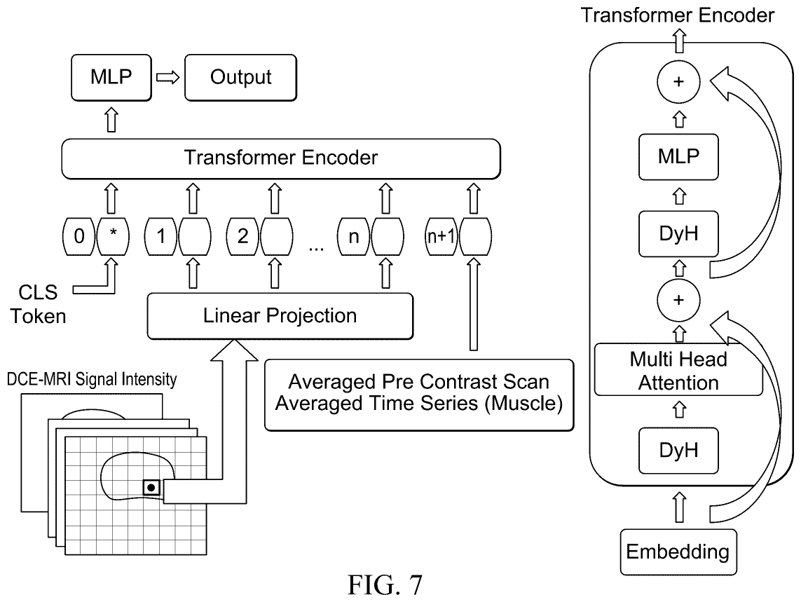

To investigate a further model where spatial and temporal features are extracted simultaneously, the disclosed subject matter further provides an exemplary ST-ViT architecture (with references to FIG. 7) for Ktrans map reconstruction. The DCE-MRI scans image data acquisition and preprocessing can be performed similarly by the MRI apparatus are analyzed as the above ST-CNN model.

The ST-ViT model processes the same 3D patches of DCE-MRI data as the one used in ST-CNN and applying linear embedding followed by a transformer encoder. A difference in the ST-ViT is to process spatial and temporal information simultaneously, which can be achieved by treating the entire 3D input (2D space+time) as a single entity for the transformer to process. Additionally, this example replaces the standard layer normalization with Dynamic TanH (DyH) activation functions, which can reduce computational complexity. Notably, this model applies linear embedding and transformer encoding to capture spatiotemporal features holistically.

### SST-VIT Model and Results

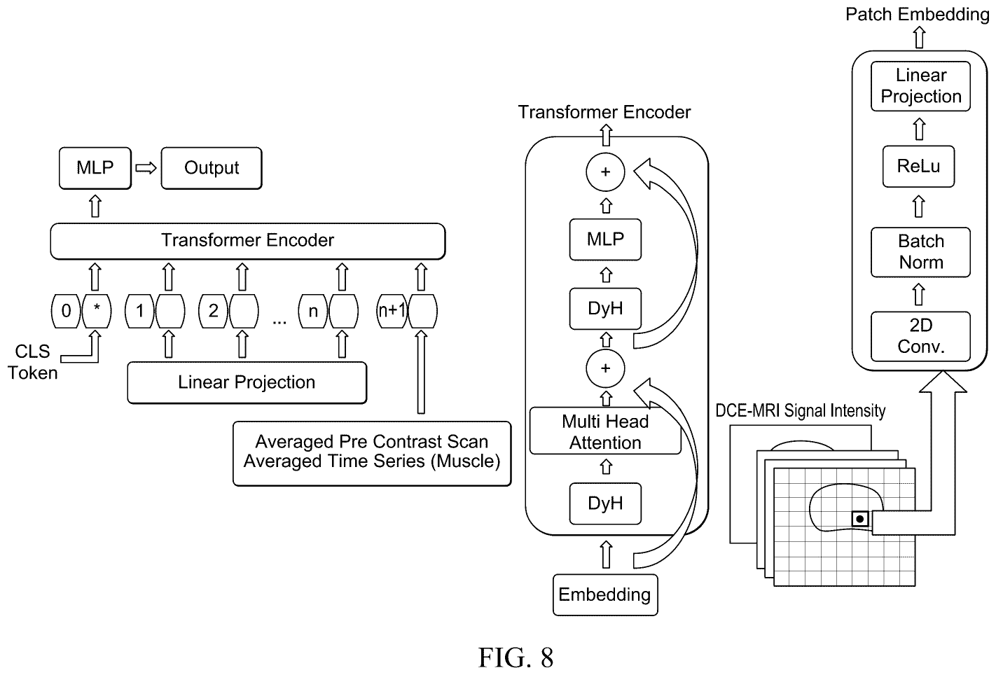

To further develop efficiency of restructuring Ktrans map, this example develops a hybrid SST-ViT model (with reference to FIG. 8), where the DCE-MRI scans image data is acquired and processed similarly as the above models.

This hybrid approach combines the strengths of CNNs for local feature extraction with the global dependency modeling capabilities of transformers. In the SST-ViT, a 2D CNN layer with kernel size 2×2, padding 1 and stride 1, first extract local spatial features from the input patch. These features, after going through 2D Batch normalization and ReLu activation are then linearly embedded and fed into a transformer encoder, which processes both spatial and temporal aspects simultaneously. Such above processes leverage CNN's efficiency at extracting local features and the ViT's ability to extract global spatiotemporal relationships. This model can be efficient in capturing local dependencies as CNNs.

### Data and Implementation

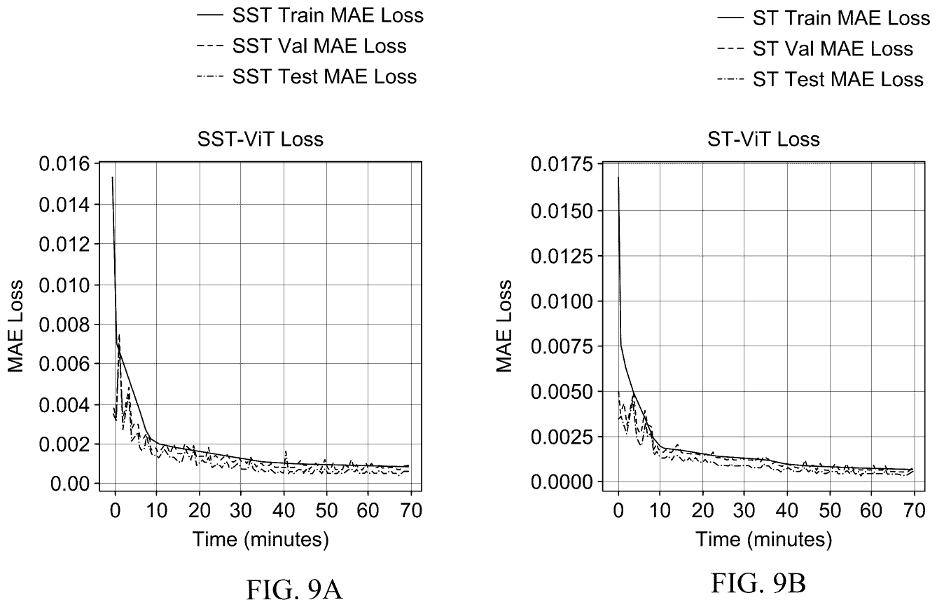

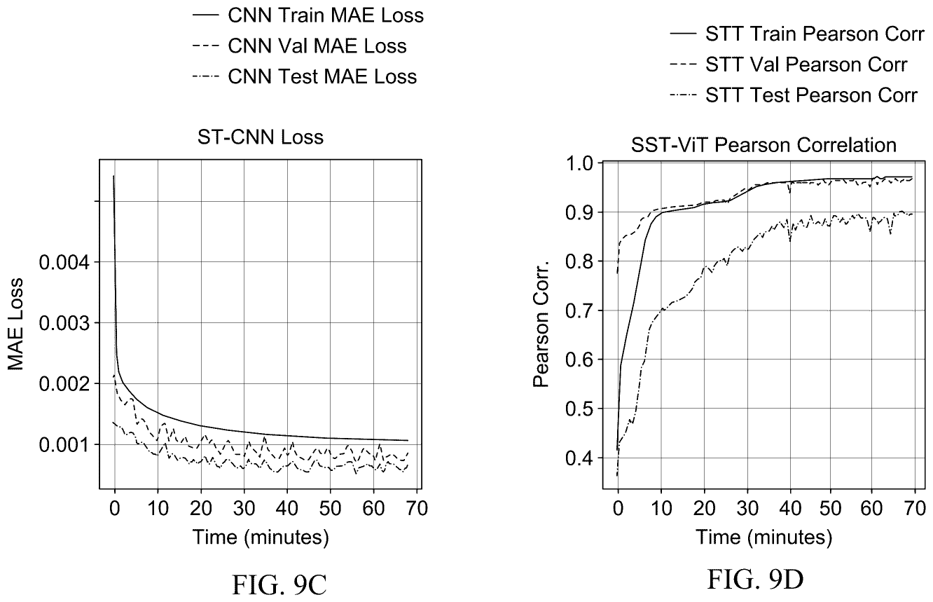

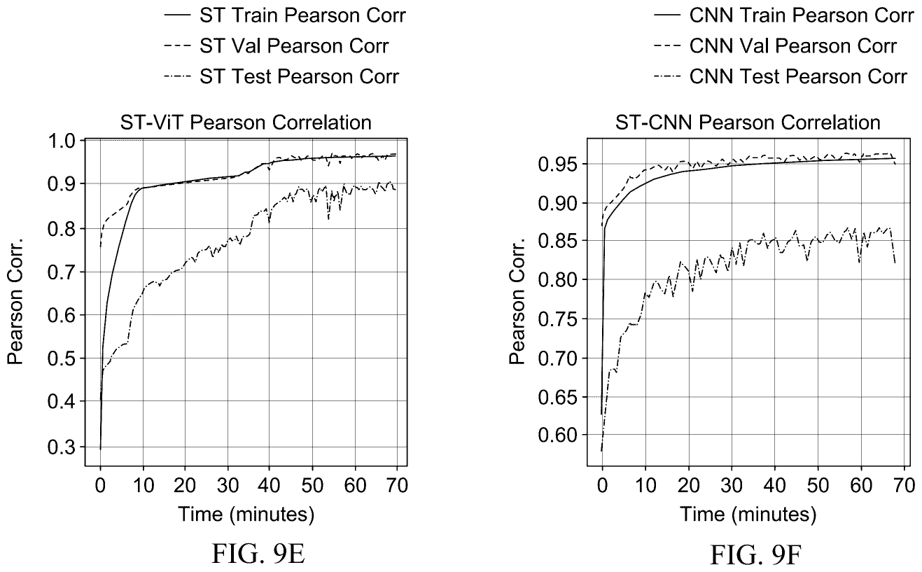

As noted above, models were implemented in PyTorch and trained with the Adam optimizer (learning rate 5e-4, batch size 256) using mean absolute error (MAE) loss, five-fold cross-validation. FIG. 9A-9F illustrates the MAE loss and the Pearson correlation curves for the three models. Both the ST-CNN and SST-ViT models converged at around 40 minutes, while the ST-ViT model took approximately 60 minutes to converge. The STCNN model achieved the lowest training loss, followed by the SST-ViT and ST-ViT models. The top plots FIG. 9A-9C show the loss and the bottom plots FIG. 9D-9F show the Pearson correlation coefficient. The x-axis represents the approximate time in minutes.

Table 3A-3C illustrate the quantitative data results of the three models for the full dose DCE-MRI scans. Notably, the ST-ViT model has a remarkable improvement in KL divergence, indicating the advantage of extracting the spatial and temporal information at once in the Kₜᵣₐₙₛ map reconstruction. Furthermore, the SST-ViT performs remarkably in all metrics, especially in SCC, NRMSE, and KL divergence, achieving the highest average in all metrics indicating its superior performance in Kₜᵣₐₙₛ map reconstruction.

TABLE 3A
ST-CNN

| ID | SCC↑ | PCC↑ | CCC↑ | NRMSE↓ | KL↓ | PSNR↑ | SSIM↑ |
|---|---|---|---|---|---|---|---|
| 1 | 0.860 | 0.994 | 0.994 | 0.097 | 0.053 | 31.42 | 0.879 |
| 2 | 0.777 | 0.993 | 0.992 | 0.108 | 0.086 | 31.33 | 0.798 |
| 3 | 0.867 | 0.994 | 0.993 | 0.102 | 0.187 | 28.98 | 0.717 |
| 4 | 0.833 | 0.991 | 0.991 | 0.121 | 0.050 | 30.49 | 0.879 |
| 5 | 0.744 | 0.943 | 0.941 | 0.306 | 0.270 | 24.60 | 0.706 |
| 6 | 0.920 | 0.977 | 0.974 | 0.182 | 0.139 | 29.12 | 0.764 |
| 7 | 0.530 | 0.972 | 0.969 | 0.239 | 0.232 | 32.65 | 0.639 |
| 8 | 0.944 | 0.982 | 0.981 | 0.142 | 0.102 | 30.47 | 0.857 |
| 9 | 0.913 | 0.983 | 0.981 | 0.158 | 0.142 | 27.86 | 0.785 |
| 10 | 0.569 | 0.945 | 0.942 | 0.319 | 0.333 | 31.39 | 0.676 |
| Ave | 0.796 | 0.978 | 0.976 | 0.178 | 0.160 | 29.83 | 0.770 |

TABLE 3B
ST-ViT

| ID | SCC↑ | PCC↑ | CCC↑ | NRMSE↓ | KL↓ | PSNR↑ | SSIM↑ |
|---|---|---|---|---|---|---|---|
| 1 | 0.826 | 0.997 | 0.996 | 0.070 | 0.014 | 34.60 | 0.862 |
| 2 | 0.777 | 0.993 | 0.993 | 0.106 | 0.035 | 31.63 | 0.868 |
| 3 | 0.941 | 0.995 | 0.995 | 0.079 | 0.015 | 31.26 | 0.919 |
| 4 | 0.798 | 0.995 | 0.994 | 0.092 | 0.011 | 33.54 | 0.891 |
| 5 | 0.904 | 0.971 | 0.971 | 0.213 | 0.026 | 27.42 | 0.865 |
| 6 | 0.964 | 0.978 | 0.978 | 0.171 | 0.005 | 30.61 | 0.924 |
| 7 | 0.533 | 0.985 | 0.984 | 0.165 | 0.020 | 37.12 | 0.908 |
| 8 | 0.914 | 0.963 | 0.961 | 0.218 | 0.038 | 27.22 | 0.806 |
| 9 | 0.910 | 0.980 | 0.979 | 0.165 | 0.039 | 27.66 | 0.837 |
| 10 | 0.599 | 0.944 | 0.943 | 0.318 | 0.041 | 34.86 | 0.904 |
| Ave | 0.817 | 0.980 | 0.980 | 0.160 | 0.024 | 31.59 | 0.878 |

TABLE 3C
SST-ViT

| ID | SCC↑ | PCC↑ | CCC↑ | NRMSE↓ | KL↓ | PSNR↑ | SSIM↑ |
|---|---|---|---|---|---|---|---|
| 1 | 0.826 | 0.996 | 0.996 | 0.077 | 0.007 | 33.21 | 0.876 |
| 2 | 0.909 | 0.995 | 0.995 | 0.085 | 0.013 | 33.54 | 0.950 |
| 3 | 0.865 | 0.995 | 0.995 | 0.082 | 0.022 | 30.93 | 0.776 |
| 4 | 0.796 | 0.995 | 0.994 | 0.099 | 0.009 | 32.60 | 0.889 |
| 5 | 0.781 | 0.984 | 0.982 | 0.169 | 0.086 | 29.21 | 0.825 |
| 6 | 0.931 | 0.981 | 0.981 | 0.157 | 0.009 | 30.62 | 0.847 |
| 7 | 0.827 | 0.981 | 0.980 | 0.193 | 0.011 | 35.90 | 0.968 |
| 8 | 0.962 | 0.983 | 0.983 | 0.141 | 0.022 | 30.29 | 0.907 |
| 9 | 0.961 | 0.990 | 0.988 | 0.122 | 0.019 | 30.53 | 0.920 |
| 10 | 0.735 | 0.950 | 0.948 | 0.301 | 0.026 | 34.81 | 0.940 |
| Ave | 0.859 | 0.985 | 0.984 | 0.143 | 0.022 | 32.16 | 0.890 |

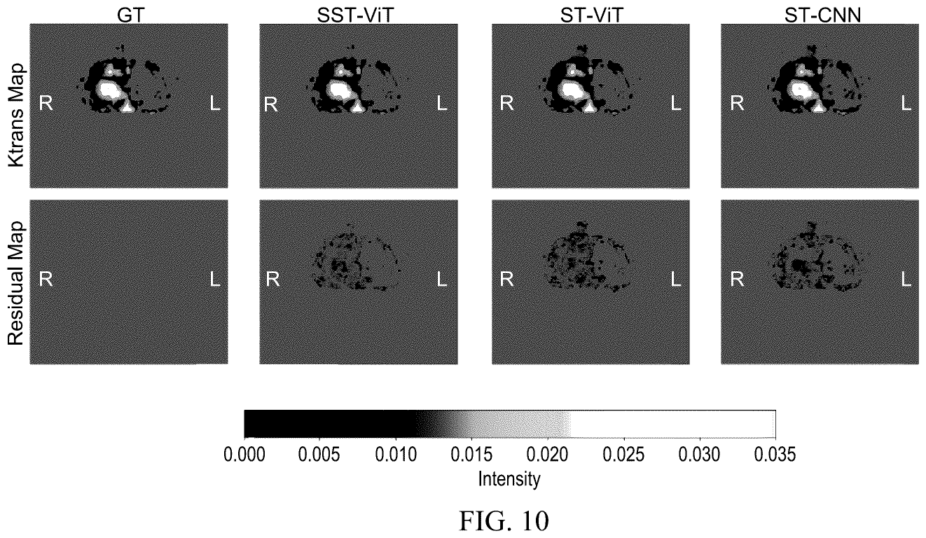

FIG. 10 illustrates performance of different models, providing predicted reconstructed Kₜᵣₐₙₛ maps and the residual maps for axial slice. From left to right, Toft's model (ground truth), (2) SST-ViT, (3) ST-VIT, (4) ST-CNN. The first row shows the reconstructed Ktrans maps, and the second row shows the residual maps. The SST-ViT outperforms both ST-CNN and ST-ViT.

These above examples related to Ktrans map reconstruction on full dose DCE-MRI scans demonstrate the performance of the disclosed subject matter, especially including hybrid SST-Vision Transformer. The SST-ViT's superior performance in both low and full dose DCE-MRI scans can be attributed to its effective combination of CNNs for local spatial feature extraction and transformers for the simultaneous extraction of spatial and temporal information, addressing the limitations of both pure CNN-based and pure transformer-based architectures, resulting in more accurate Kₜᵣₐₙₛ map reconstruction. Further, hybrid CNN Transformer architectures provide an effective balance between local spatial and simultaneous spatial and temporal extraction. The disclosed SST-ViT model enables efficient and accurate Kₜᵣₐₙₛ map reconstruction, which can be important for the clinical translation of FUS-mediated BBB opening treatments.

### Medical Imaging System

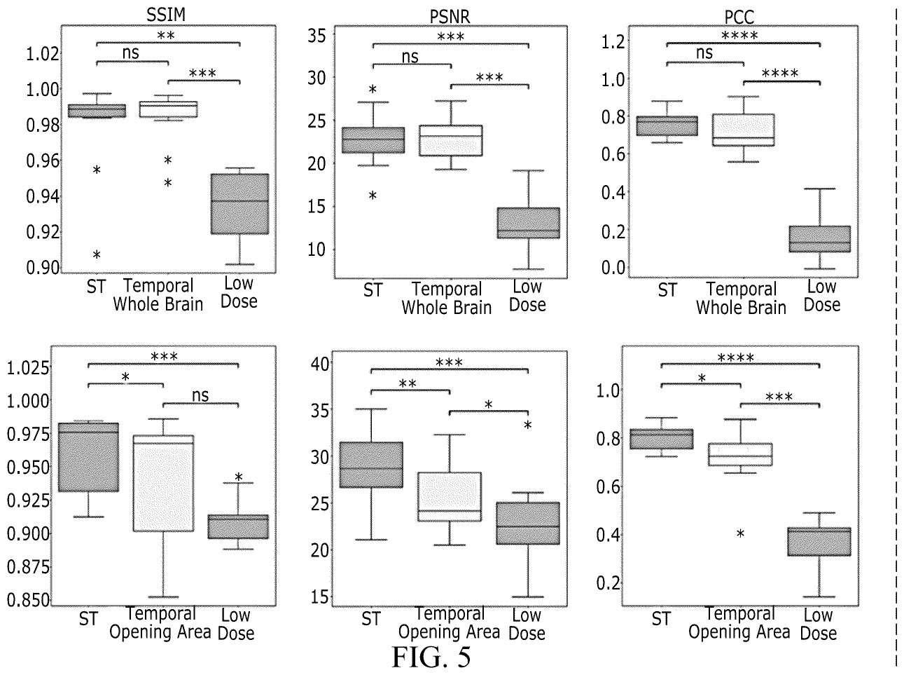

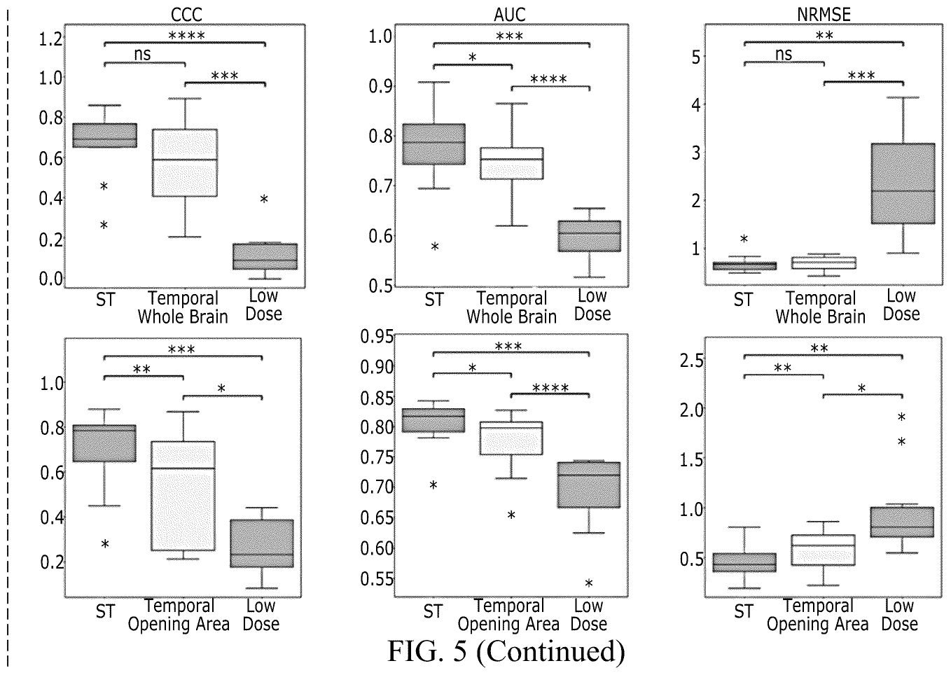

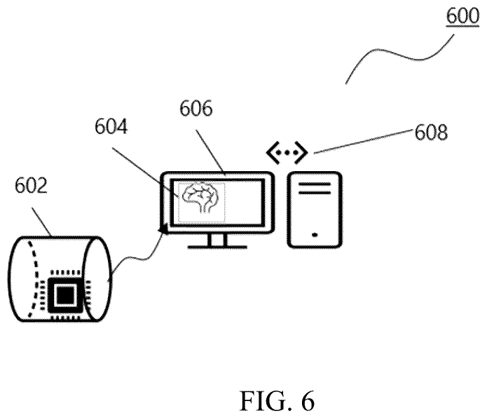

In certain embodiments, the disclosed subject matter further provides a medical imaging system integrating the above-described deep learning method for DCE-MRI scans. Referring now to FIG. 6, a detailed representation of an exemplary medical imaging system is presented. Such a medical imaging system 600 includes a DCE-MRI apparatus 602 configured to obtain a plurality of DCE-MRI images 604; and a processing unit 606 configured to implement a deep learning method 608 for analyzing the plurality of DCE-MRI images 604 to output a corresponding plurality of Kₜᵣₐₙₛ. The processing unit 606 can be a computing system equipped with a multi-core processor, dedicated graphics processing unit (GPU), and sufficient memory to efficiently execute the complex computations involved in the deep learning method.

In certain embodiments, the medical imaging system further comprises a display unit configured to present a Kₜᵣₐₙₛ map on visual representations of the plurality of Kₜᵣₐₙₛ. The display unit can include a high-resolution monitor with color accuracy, providing a clear and detailed visualization of the imaging data and the plurality of Kₜᵣₐₙₛ.

In certain embodiments, the DCE-MRI apparatus can be further configured to adjust imaging parameters based on the plurality of Kₜᵣₐₙₛ. For example, the DCE-MRI apparatus can include a user interface allowing radiologists or technicians to interact with the medical imaging system, modifying imaging parameters such as scan duration, sequence parameters, or field of view based on the real-time analysis of the deep learning method.

In certain embodiments, the processing unit is further configured to store the plurality of Kₜᵣₐₙₛ in a storage device for subsequent analysis. The storage device can include a high-capacity, high-speed storage medium such as solid-state drives (SSD) or network-attached storage (NAS), ensuring rapid access to the Kₜᵣₐₙₛ values for further research, comparisons, or archiving.

In certain embodiments, a FUS apparatus is further integrated into the DCE-MRI apparatus for inducing BBB-opening. The FUS apparatus can include transducers and control systems capable of precisely targeting and applying focused ultrasound to induce controlled BBB opening during the imaging procedure, enhancing the system's capabilities for both imaging and therapeutic applications.

It will be understood that the foregoing is only illustrative of the principles described herein, and that various modifications can be made by those skilled in the art without departing from the scope and spirit of the disclosed subject matter. For example, the system and methods described herein are used for opening the BBB of a subject. It is understood that that techniques described herein are useful for opening of other tissues. Further, the techniques described have been performed on mice in certain embodiments, but it is understood the techniques are applicable to other subjects, such as humans. Moreover, the features in embodiments described herein can be combined and/or rearranged to create new embodiments. For instance, the type of software, the order sequence of spatial and temporal network, and the numbers of CNN encoders are adjustable and expandable within the knowledge of person skilled in the art.

We claim:

1. A deep learning method for reducing dosage of Gadolinium-Based Contrast Agents (GBCAs) in medical imaging, comprising:
a. applying dynamic contrast-enhanced magnetic resonance imaging (DCE-MRI) to a subject to obtain a plurality of DCE-MRI images;
b. analyzing the plurality of DCE-MRI images with a deep learning model using a spatiotemporal network to obtain a corresponding plurality of volume transfer constants (Kₜᵣₐₙₛ); and
c. forming a map using the plurality of Kₜᵣₐₙₛ.

2. The method of claim 1, wherein the analyzing the plurality of DCE-MRI images comprises:
extracting spatial information using a three-dimensional convolutional neural network (CNN) encoder.

3. The method of claim 2, wherein the analyzing the plurality of DCE-MRI images further comprises:
concatenating the spatial information with two reference arrays, including average intensity of pre-contrast images and average DCE-MRI time series signal.

4. The method of claim 3, wherein the analyzing the plurality of DCE-MRI images further comprises:
implementing a temporal network, comprising a one-dimensional CNN layer to blend spatial and reference information, and two separate CNN pathways capturing long-term and short-term temporal characteristics.

5. The method of claim 4, wherein the analyzing the plurality of DCE-MRI images further comprises:
fusing long-term and short-term temporal characteristics for outputting, using additional one-dimensional CNN layers and a fully connected layer.

6. The method of claim 1, wherein the analyzing the plurality of DCE-MRI images further comprises:
applying a Leaky Rectified Linear Unit (ReLU) activation.

7. The method of claim 1, wherein the deep learning model is configured to be trained in a dataset employing BBB-opening patches.

8. The method of claim 1, wherein the applying DCE-MRI comprises:
inducing focused ultrasound with administration of microbubbles to BBB-openings.

9. The method of claim 8, further comprises injecting contrast agents to a trace of the BBB openings.

10. The method of claim 1, wherein the analyzing the plurality of DCE-MRI images comprises processing spatial and temporal information simultaneously by treating a three dimensional input as a single entity for a CNN encoder.

11. The method of claim 10, wherein the analyzing the plurality of DCE-MRI images comprises processing three dimensional patches of the DCE-MRI data and applying linear embedding followed by the CNN encoder to capture spatiotemporal features.

12. The method of claim 1, wherein the analyzing the plurality of DCE-MRI images comprises extracting local spatial information from input patches, followed by a CNN encoder to capture global spatiotemporal relationships.

13. The method of claim 9, wherein the contrast agents are injected at two times

14. The method of claim 1, wherein the Kₜᵣₐₙₛ map is formed through a general kinetic model (GKM) model.

15. The method of claim 8, wherein the employing BBB openings patches comprises cropping each voxel of Whole Brain (WB) scan into patches for extracting spatial information.

16. A medical imaging system integrating a deep learning method, comprising:
a dynamic contrast-enhanced magnetic resonance imaging (DCE-MRI) apparatus configured to obtain a plurality of DCE-MRI images; and
a processing unit configured to implement the deep learning method of claim 1, for analyzing the plurality of DCE-MRI images.

17. The medical imaging system of claim 16, further comprising a display unit configured to present a Kₜᵣₐₙₛ map on visual representations of the plurality of Kₜᵣₐₙₛ.

18. The medical imaging system of claim 16, wherein the DCE-MRI apparatus is further configured to adjust imaging parameters based on the plurality of Kₜᵣₐₙₛ.

19. The medical imaging system of claim 16, wherein the processing unit is further configured to store the plurality of Kₜᵣₐₙₛ in a storage device for subsequent analysis.

20. The medical imaging system of claim 16, wherein a focused ultrasound apparatus is further integrated to the DCE-MRI apparatus for inducing a BBB-opening.
